(19) United States
(12) Patent Application Publication
(10) Pub. No.: US 2006/0122509 A1
(43) Pub. Date: Jun. 8, 2006

(54) System and methods for destroying adipose tissue

(75) Inventor: Charles S. Desilets, Edmonds, WA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: LipoSonix, Inc., Bothell, WA (US)

(21) Appl. No.: 11/286,042

(22) Filed: Nov. 23, 2005

Related U.S. Application Data
(60) Provisional application No. 60/630,857, filed on Nov. 24, 2004.

(51) Int. Cl. A61B 8/12

(52) U.S. Cl. 600/439; 601/2

(57) ABSTRACT

Methods and systems for the destruction of adipose tissue are disclosed. A method is provided for creating a surface map corresponding to a volume of adipose tissue for noninvasive treatment, and additional methods are provided for the treatment of the adipose tissue.

### CROSS-REFERENCES TO RELATED APPLICATIONS

The present application is a non-provisional of U.S. Patent Application Ser. No. 60/630,857 (Attorney Docket No. 021356-001400US), filed Nov. 24, 2004, the full disclosure of which is incorporated herein by reference.

All publications and patent applications mentioned in this specification are herein incorporated by reference to the same extent as if each individual publication or patent application was specifically and individually indicated to be incorporated by reference

### BACKGROUND OF THE INVENTION

1. Field of the Invention

The present invention relates to systems and methods for the destruction of adipose tissue (fat).

2. Description of the Prior Art

Body sculpting has developed into a highly sought after procedure for reducing a person's adipose tissue and restoring people to a leaner, trimmer physique. The field of cosmetic surgery has ballooned considerably with developments in both tools and techniques. One of the more popular procedures for both quick reduction in adipose tissue volume and body sculpting is liposuction.

Liposuction is a method of body contouring that can dramatically improve the shape and contour of different body areas by sculpting and removing unwanted fat. More than 500,000 liposuction procedures are performed annually. Recent innovations and advances in the field of liposuction include the tumescent technique and an ultrasonic assisted technique. Traditional liposuction was done by making small incisions in desired locations, then inserting a hollow tube or cannula under the skin and into the fat layer. The cannula is connected to a vacuum and the fat is vacuumed out under high suction pressure. This procedure indiscriminately removed fat, connective tissue, blood vessels and nerve tissue. The procedure caused bleeding, bruising, trauma, and blood loss, restricting the amount of fat removal possible.

The Tumescent technique allows for removal of significantly more fat during the operation with less blood loss. Tumescent liposuction injects a fat layer with large amounts of saline and adrenalin solution before suctioning. A cannula is again used with a suction device to remove fat. This procedure reduces the bleeding of traditional liposuction. However the procedure still removes a significant amount of structural tissue, blood and nerve tissue.

The most recently approved innovation is Ultrasound Assisted Lipoplasty (UAL). UAL utilizes a titanium cannula that has the tip vibrating at ultrasound frequency. This vibration disrupts the near volume fat cells and essentially liquefies them for easy removal. UAL uses a low power suction and draws the fat material only in the near vicinity of the cannula tip. This technique is more refined and gentle to the tissues, compared to traditional surgical liposuction and there is less blood loss, less bruising, less pain, and a significantly faster recovery period for the patient.

The use of ultrasound for surgical procedure is not restricted to UAL. High intensity focused ultrasound (HIFU) techniques have been employed by others for cancer therapy.

### BRIEF SUMMARY OF THE INVENTION

Provided herein are methods for destroying adipose tissue in association with a noninvasive cosmetic surgery procedure. In one embodiment, there is provided for a method for projecting a volume of tissue onto a skin surface in preparation for a noninvasive cosmetic therapy procedure. The method has the steps of determining a volume of tissue suitable for a noninvasive cosmetic therapy procedure, and creating a surface area map corresponding to the volume of tissue on a skin surface. The surface map provides sufficient volumetric information to guide a user in conducting the noninvasive cosmetic therapy procedure.

In a second embodiment, a method for initiating a reduction in a volume of adipose tissue comprises the step of moving a therapeutic high intensity ultrasound transducer over a patient skin surface while emitting high intensity ultrasound into a volume of adipose tissue such that a biological response is initiated that leads to a reduction in said volume of adipose tissue.

In a third embodiment, a method for reducing a volume of adipose tissue in a patient comprises the steps of moving a high intensity focused ultrasound transducer over a skin surface, and irradiating a volume of adipose tissue below the skin surface using the high intensity focused ultrasound transducer. The energy deposited can be determined by an energy flux (E_{f}) value, which should be at least 35 J/cm².

In yet another embodiment, a method for destroying adipose tissue uses high intensity focused ultrasound. The method comprises the steps of determining a volume of adipose tissue to be treated, marking out a corresponding surface area of skin, dividing the surface area into a plurality of individual treatment sections, and applying therapeutic ultrasound energy to one section of the plurality of individual treatment sections with an ultrasound transducer until sufficient energy has been deposited to at least partially destroy the adipose tissue. Usually, additional treatment sections will be treated successively.

In still another embodiment there is a system for coupling a high intensity focused ultrasound transducer to a patient. The system has at least the following components: a fluid circuit, pump, vacuum chamber, filter and fluid reservoir. The fluid circuit conveys a coupling fluid. There is a pump for circulating the coupling fluid through the circuit and a vacuum chamber. The vacuum chamber removes dissolved gasses from the coupling fluid. A filter is used for removing particulate matter. There is also a coupling fluid reservoir connected to the fluid circuit for coupling a transducer to a patient.

### BRIEF DESCRIPTION OF THE DRAWINGS

FIG. 1 illustrates an tissue sample showing a single line of therapy treatment.

FIG. 2 illustrates a tissue sample with a cross section view of a continuous scan line.

FIG. 3 shows a cross section a scan line made up of discrete lesion fields.

FIG. 4 illustrates a jumping pattern of lesion fields.

FIGS. 5A-C provide various examples of lesion field patterns.

FIG. 6 provides a schematic view of a system having a fluid coupling circuit.

### DETAILED DESCRIPTION OF THE INVENTION

Described herein are systems and methods for non-invasive cosmetic therapies such as the reduction of adipose tissue volumes in a patient. The system described herein uses a therapeutic ultrasound transducer, such as a high intensity focused ultrasound (HIFU) transducer, to achieve a desirable body contouring effect. The therapy methods and system described obtained desirable results without severe adverse side effects, such as hazardous long term systemic or local effects, nor any other serious side effects of the therapy procedures described herein. Desirably, the out come of the therapy procedure disclosed herein is a reduction of the volume of adipose tissue in patients undergoing the therapies described, as well as a reduction in the girth of those patients. Modest side effects including mild transient skin redness (erythema) are acceptable during the course of the procedures detailed herein.

The procedures described herein are able to treat nearly any volume of tissue. As a pretreatment procedure, there is a method for projecting a volume of tissue onto a skin surface in preparation for the noninvasive cosmetic therapy procedure. The method has the steps of determining the volume of tissue suitable for the noninvasive cosmetic therapy procedure, and creating a surface area map corresponding to the volume of tissue. The surface area map is projected or otherwise formed on the skin surface, and provides sufficient volumetric information to guide a user in conducting a noninvasive cosmetic therapy procedure.

In general, cosmetic therapy procedures are known and used for body sculpting, or body contouring. Currently liposuction is the method of choice for use in these cosmetic therapy procedures. However liposuction is an invasive procedure and its draw backs are well known. A noninvasive cosmetic therapy procedure desirably achieves similar results as liposuction, without the accompanying risks and detriments of an invasive procedure.

The creation of a surface area map corresponding to a volume of tissue beneath the skin is desirable so a user of a noninvasive device, can perform the noninvasive therapy procedure with a level of safety and confidence that is practiced in invasive procedures. In the treatment of adipose tissue, the depth and boundaries of the tissue are desirable known so the user has a good idea of the physical boundaries or limits to the treatment he or she provides to the patient. Adipose tissue volume can be detected using an imaging device, such as ultrasound or MRI. Users may also use physical tests for determining adipose tissue volumes (such as a pinch test or caliper test) and rely on their experience and judgment to interpret the physical tests. Once the user has a sense for the tissue volume under the skin, the user can create the surface area map.

The surface area map can be drawn onto the patient's skin or projected on to the skin, or in any suitable manner laid out so during the noninvasive cosmetic therapy procedure, the user knows where the boundaries of the tissue to be treated are. The user can create a simple boundary map to show the length and breadth of the adipose tissue layer she wishes to treat. Alternatively the user may create a series of contour lines that will provide depth information when examining the surface area map. In another embodiment, the surface area map may be further partitioned into a series of purposely sized shapes that correspond to the foot print of a noninvasive therapy device. This will enable the user to line up the foot print of the noninvasive therapy device with the individual partitions (individual treatment sections) and carryout the treatment going from one individual treatment section to the next.

The surface map described above is well suited to be used in combination with a non-invasive therapy device, such as a high intensity ultrasound device, to perform a non-invasive cosmetic therapy procedure.

One such cosmetic therapy method involves the use of a system preciously described in co-pending U.S. patent application Ser. No. 11/026,519; entitled “Systems and Methods for the Destruction of Adipose Tissue” filed on Dec. 29, 2004. In a first method of the present invention, there is a method for initiating a reduction in volume of adipose tissue. The method has the step of moving a therapeutic high intensity focused ultrasound transducer (transducer) over a patient skin surface while emitting high intensity ultrasound into a volume of adipose tissue, such that a biological response is initiated that leads to a reduction in the volume of adipose tissue.

In this embodiment, the ultrasound transducer deposits sufficient energy to initiate a biological response, however the energy deposited is not sufficient to have the effect of killing or destroying adipose tissue through the application of ultrasound by itself. This method allows for the use of ultrasound to cause disruption or irritation of the local tissue the ultrasound energy is focused into, so that the patient's body will respond with a mild wound healing response. The wound healing response may be a protein chain coagulation or poreation of cellular membranes within the adipose tissue. So long as the ultrasound produces some reaction in the tissue that can cause the tissue volume to be reduced.

The transducer may be a classically focused transducer, having a bowl like shape and forcing the convergence of ultrasound energy into a focal zone, or it may be a partially focused ultrasound transducer as previously described co-pending U.S. patent application Ser. No. 10/816,197; entitled “Vortex Transducer” and filed on Mar. 31, 2004. Reference herein to HIFU includes the use of partially focused high intensity ultrasound as well as traditionally focused high intensity ultrasound transducers.

In order to treat a volume of adipose tissue, it is desirable to cause the transducer to be moved over the surface area map of the adipose tissue, while emitting HIFU energy. The transducer can be moved across the surface in a scanning mode, or a jumping mode. A scanning mode can be a continuous motion, like traversing one end of an individual treatment section to another, or moving in an arch or similar fashion. The sweeping motion of the transducer does not equate to the transmission pattern of the transducer itself, but merely to the type of motion the transducer undertakes during the non-invasive cosmetic therapy procedure. Thus the transducer may produce both continuous or discrete lesion fields while traveling across the skin surface in continuous sweeps.

A jumping mode is achieved when the movement of the transducer is discrete and caused to pause to produce individual lesion fields. The discrete motion may not be perceptible to the human eye, as the motion of the transducer may be machine controlled as previously described in co-pending U.S. patent application Ser. No. 11/027,912; entitled “Ultrasound Therapy Head with Movement Control”, filed on Dec. 29, 2004. The emission of ultrasound energy into the patient's adipose tissue will produce some kind of lesion field. When using the method described above for initiating a reduction in the patient's adipose tissue volume, the lesion field may not be immediately apparent.

In another embodiment there is a method for reducing a volume of adipose tissue in a patient having the steps of moving a HIFU transducer over a skin surface and irradiating a volume of adipose tissue below the skin surface using the HIFU transducer such that the transducer deposits an energy flux value of at least 35 J/cm². In this method the reduction of adipose tissue is generated from a combination of effects. One of the effects of the ultrasound energy is the destruction of adipose tissue (or the necrosis of adipose tissue). Once the adipose tissue is destroyed, a wound healing response is triggered in the patient so that the dead or destroyed cells, interstitial matter and other materials affected by the HIFU energy are removed from the body by the patient's natural healing process. The volume of tissue to be treated may cause the user to increase the energy flux, or alter other parameters of the energy flux to achieve the desired results. The transducer may be capable of an E_{f} value up to 456 J/cm².

The absorption of HIFU energy in matter can produce a lesion field. The lesion field is the volume of matter that absorbs the HIFU energy, and is effected by that energy. In a patient, the lesion field corresponds to the volume of tissue disrupted through either thermal or mechanical effects resulting from the focused HIFU energy in the tissue. If the transducer is held stationary, the HIFU energy can produce a single lesion field. If the transducer is moved the HIFU energy may produce a lesion field that in continuous. One may imagine, for purposes of analogy only, a magnifying glass focusing sunlight on a wooden board. If the magnifying glass is held stationary, a single spot is affected. Depending on the amount of sun light (intensity) and the length of time the magnifying glass is focused on that one spot, the wood may become warm, brown, black or even catch fire. If the magnifying glass is moved, so that the focused sunlight travels over the board, a trail of the focus effect is created. The trail of the focused sunlight may be merely warm to the touch, or it may brown, blacken or catch fire. If the magnifying glass is moved from one spot to another on the board without focusing sunlight on the board, then discrete focal effects will be observed with no change in the board between the discrete focal points.

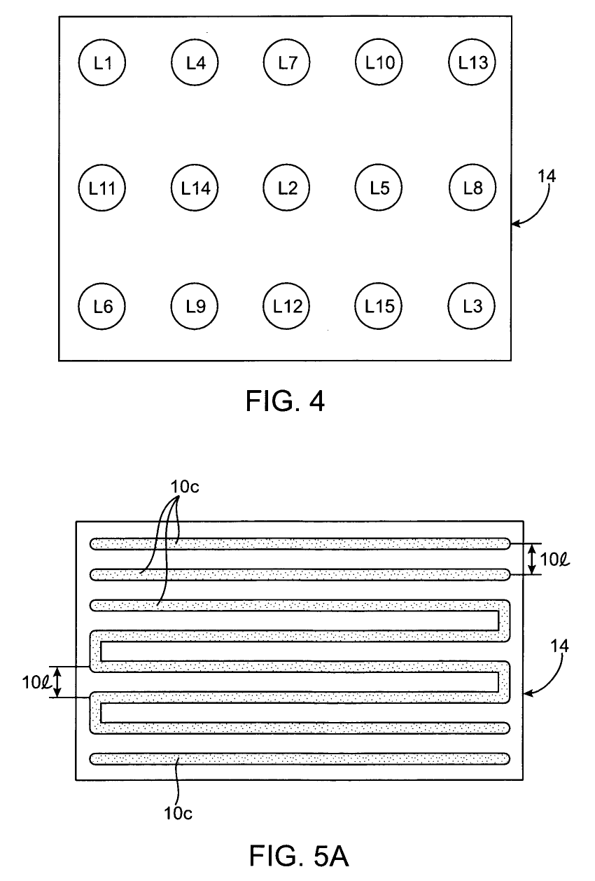

Similarly now with the HIFU transducer, the HIFU energy may be on continuously and sweep a path through the tissue, or it may be on incrementally to create discrete lesion fields. If the transducer is physically moved from one place to another in sequence, this is physical jumping of the transducer. If there is a time delay between the creation of one of the lesion fields and an adjacent lesion field, there is a time delay or temporal jumping of the transducer. The two effects can be combined to produce lesion field patterns involving both physical and time delay jumping. An example of combined spatial and temporal jumping is shown in FIG. 4. Fifteen discrete lesion fields are shown in a single treatment section 14. The discrete lesion fields are made sequentially from L1 to L15 and spaced apart as indicated. The discrete lesions are spaced apart from each other (as one sees that lesion L1, then L2 and so on) while there is some time delay between adjacent lesions (There is enough time between adjacent lesions L1 and L4 for two other lesions to have been formed).

The treatment volume is limited by the surface area that the transducer can cover during a therapy procedure. During the course of a therapy procedure it is possible to treat between 500 to over 900 cc of adipose tissue in a single session. It may be desirable to treat even larger volumes by adjusting the parameters of the therapy and system, so that the transducer moves at a higher velocity, while still maintaining an effective and desirable energy flux (or energy output). The transducer used may also include multiple transducers (as previously described in co-pending U.S. patent application Ser. No. 11/027,919; entitled “Component Ultrasound Transducer,” and filed on Dec. 29, 2004) driven at the same time to increase the treated volume in a given treatment session. Small volumes of adipose tissue may be treated going down to a single cc of volume, up to more than 1500 cc.

A range of energy flux values can be used to obtain the desired results. Variables in the procedure depend in large part by the amount of time a patient has to undergo the therapy methods described, as well as the volume the patient wishes to have treated. Patients having a small amount of tissue to be treated during a session may take advantage of a therapy method that allows for the transducer to move slowly while emitting a lower amount of energy during the procedure, while patients desiring to have a large volume of tissue treated in the same time period will need a faster scan rate on the transducer, and a correspondingly higher energy output in order to achieve the desired results. The E_{f} (see below) during these two very different therapy sessions may range from 35 J/cm² to 456 J/cm².

The user may create a surface map to follow during a therapy procedure, or she may rely on an alternative manner to provide a noninvasive tissue destroying therapy in a safe manner (such as using a depth detector, like an “A” line scan, in combination with the HIFU transducer). Once the boundaries and depths of the tissue volume have been identified, it is desirable that a coupling gel or other coupling agent be used to couple the transducer face to the patient. An acoustic gel or coupling agent is desirably degassed, and massaged on to the patient's skin to minimize air bubbles that may form in the imperfections of the skin, hair follicles and/or sweat glands. Desirably the skin surface has been pre-washed and is clean of most particulate matter. To reduce or eliminate particulate matter that may be contributed by the user, gloves or other tools may be used to massage the coupling agent onto the patient.

After the coupling agent is properly placed onto the patient, the user can place the ultrasound transducer onto the patient. The user desirably exercises sufficient caution so the transducer is placed on the skin surface without trapping air between the transducer and the coupling agent. The transducer desirably is capable of moving according to a preset program providing for the transducer to sweep back and forth and irradiate the adipose tissue with ultrasound according to the user's desire. The transducer may be placed within a therapy head having a motor assembly so the transducer moves within the therapy head, or the transducer may be set up on a mechanical arm or other device that moves the transducer during the procedure. Once the transducer is placed in the proper position to begin therapy, the transducer is activated and the movement of the transducer begins.

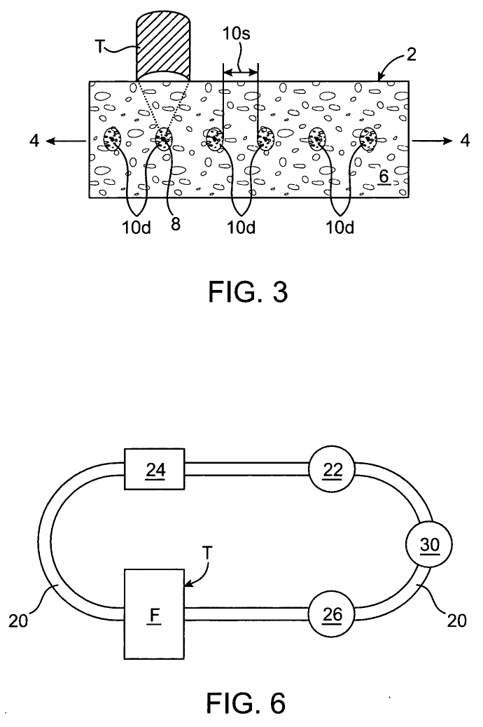

If the ultrasound transducer is mounted in a housing with a motor control, or the transducer is attached to a motorized mechanism, then the transducer can be moved through electronic control to provide treatment. The movement mechanism the transducer is connected to may be programmed with such information as the velocity, line spacing, or patterns of movement to correspond with the treatment type. The basic use of the transducer involves simply having the transducer placed over a single location without use of any motor controls and activating the transducer over a single spot on the skin surface. If the transducer is left to focus on a single spot, a discrete lesion field 10d will be formed. Multiple lesion fields may be created along a scan line 4 by jumping the transducer from one focal zone to the next, and produce a new lesion field at each new position (FIG. 3).

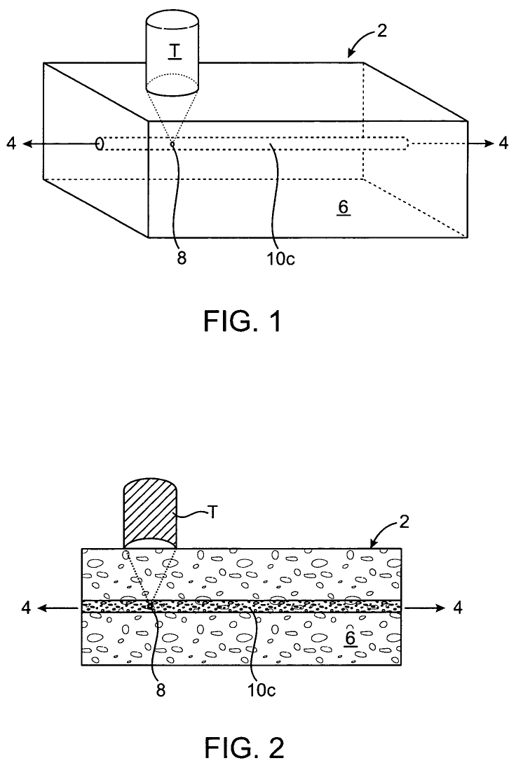

One example of a simple motion is single linear path of the transducer over the patient's skin surface as shown in FIG. 1. The HIFU transducer T is shown on the patient skin surface 2. The HIFU energy is focused at a focal zone 8, and the transducer can move in a linear path that creates a single scan line 4. The transducer T is shown moving over a volume of adipose tissue 6. The treatment volume is defined by either a discrete lesion field 10d, or a continuous lesion field 10c. Discrete and continuous lesion fields maybe created contiguously in the adipose tissue.

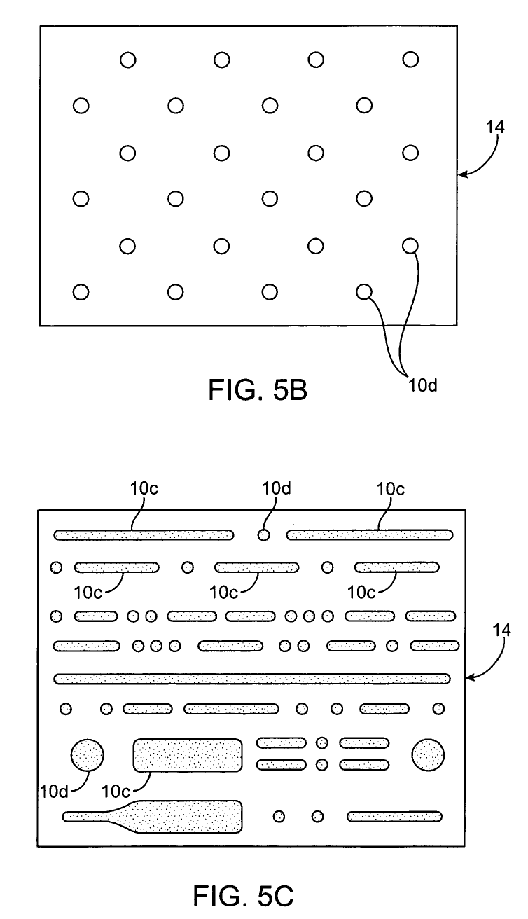

FIG. 2 provide a cross section view of the adipose tissue 6 in FIG. 1. In this cross section view, a continuous lesion field 10c is shown as the transducer T is moved across the patient skin surface 2 along the scan line 4. If the transducer is moved back and forth to produce multiple scan lines in a pattern similar in motion to a raster scan, then the scan lines can form a series of parallel lesion fields within a treatment section 14 (FIG. 5A). The practice of placing parallel scan lines close together allows for thermal energy build up in one scan line to affect the amount of tissue affected in the adjacent scan line. The distance between parallel scan lines is the line spacing 101 between contiguous lesion fields. The interaction between the scan lines is a cooperative effect. The cooperative effect may increase the accumulation of thermal energy in the adipose tissue generated by the ultrasound transducer. In some therapy methods, this cooperative effect may be desirable, while in other therapy methods it may be undesirable. The E_{f} the adipose tissue experiences can be altered by having a high power sweep moving quickly and with close scan lines, verses a low power sweep moving at the same speed and having a larger distance between scan lines.

The treatment section 14 is a defined space, such as a square or rectangle. The treatment section may correspond to the transmission window of a therapy head having a movement control, alternatively the treatment section may correspond to the range of motion of a robotic mechanical arm. The movement of the transducer continues until the transducer has moved over the entire defined space. Note—the defined space or treatment section may be the entire area of the surface area map or marked area.

The transducer is desirably simultaneously emitting ultrasound energy as it moves. The transducer may operate in continuous wave mode, such that ultrasound is constantly emitted from the transducer during the entire time period of the scan, or it may operate in a pulse wave mode, so that the transducer emits ultrasound energy in discrete pulses while moving. The movement speed will dictate whether the focal zones of the transducer are positioned in a continuous series, or as a set of dashed focal zones in space (one might imagine the therapy treatment to distribute the emitted focal zones as a string of Morse code dots or dashes, shown in alternating lines in FIG. 5C). The combination of discrete lesion fields 10d and continuous lesions fields 10c shown in FIG. 5C do not indicate any special operation or effect. The combination of different lesion fields is merely illustrative that any combination of discrete and continuous lesion fields may be used in a treatment section. If the transducer follows a raster scan pattern, then the emission pattern may have dots or dashes perpendicular to the parallel travel lines as the transducer moves incrementally from one scan line to the next.

Alternatively the transducer may be moved in a linear scan pattern where the transducer emits energy while traveling one direction, but not the other. Additional patterns are possible and depend only on the motion capabilities of the motor(s) driving the transducer movement. Likewise a scan pattern of ultrasound energy may follow any pattern of the transducer's movement, with emission corresponding to any combination of on/off time that the system may be programmed with. Discrete lesion fields may be arranged to form a series of cells in the tissue (FIG. 5B) while preserving the integrity of the tissue by having some lesion field spaces 10s.

The transducer may create enlarged lesion fields, or thermal dosage fields by placing scan lines close together.

The movement of the transducer can be set up so the transducer skips one or more lines in the scan pattern, and then comes back to do those scan lines later, or the transducer can be programmed for repetitive motion over the same scan lines. The transducer motion may be altered to create a first raster scan with scan lines in one direction, and then a second raster scan with scan lines perpendicular to the first pattern. The second raster scan may have any orientation with regard to the first, and there is no limit to the number of repeat scans over the same area.

In any of the embodiments described herein, the instrument parameters may be varied or compensated for to allow a substantially constant E_{f} value during a procedure. Similarly, the instrument parameters may be adjusted to utilize different or variable E_{f} values during a single procedure.

Any therapy system capable of matching the parameters described herein may be suitable for use with the methods described. Generically, the energy flux for the destruction of adipose tissue is desirably above 30 J/cm2/sec. More desirably is an E_{f} value between 35 and 200 J/cm². The E_{f} value for a raster scanned treatment volume is defined by the following equation:
E_{f}=[(p×(l/v)×duty cycle)×(nl)]/sa

wherein

p=power

l=line length

v=velocity

dc=duty cycle

nl=number of lines

and

sa=scanned area.

The E_{f} value for a spot treated volume is defined by the following equation:
E_{f}=[(p×(tₒₙ)×duty cycle)×(np)]/sa

wherein

p=power

tₒₙ=time on

dc=duty cycle

np=number of points

and

sa=scanned area.

The procedures used to validate the E_{f} formula in the present description relied principally on high intensity ultrasound energy. The frequency range for the ultrasound transducer varies from 200 kHz to 6 MHz, though there is latitude in the therapy methods described to use even higher frequencies if desired for certain areas of the body. The general frequency range is from 2 MHz to 4 MHz.

The various parameters utilized in establishing the methods herein include power ranging between 100 to 378 watts (acoustic) inclusively with a pulse repetition frequency (PRF) of 1 to 10 kHz. Desirably the PRF is about 5 kHz. The duty cycle of the transducer may be less than 100% (PW mode) or 100% (CW mode). The burst length may be continuous (CW mode) or pulsed (PW mode) with the burst length varying from about 5 μsec to 15 μsec. The transducer is also designed to be moved, either manually or mechanically, and the scan rate may vary from 1 mm/sec to 30 mm/sec. Desirably the sweep velocity is from 4 to 25 mm/sec. Individual lines of therapy are spaced between 1 and 10 mm apart. Line spacing can be adjusted to promote cooperative therapy effects between lines (2 mm or less) or to reduce cooperative effects by increasing the line spacing (3+mm).

The many parameters described may be used in combination to tailor a non-invasive cosmetic therapy procedure to a patient's particular desires, or a desired clinical outcome. Another embodiment of the present invention makes use of the combination of the many elements described. The method comprises the steps of determining a volume of adipose tissue to be treated and marking out a corresponding surface area of skin. The marked surface area can be a surface area map having sufficient detail volumetric detail to assist a user in carrying out a non-invasive therapy procedure. However the marked surface area need not have that level of detail if the user has some other method of providing depth and boundary information. Once the surface area is marked, the surface area is divided into a plurality of individual treatment sections. Then HIFU energy is applied to one section of the plurality of individual treatment section with an ultrasound transducer until sufficient energy has been deposited to at least partially destroy the adipose tissue.

The manner of applying the therapeutic ultrasound energy may involve moving the HIFU transducer in a manner such that sequential application of ultrasound energy are spaced apart to non-adjacent sections. Alternatively there may be a timing delay in the treatment of physically adjacent sections.

In another embodiment the transducer may be moved in a fashion so that the application of therapeutic ultrasound energy involves scanning the transducer over a treatment surface area at a velocity and line spacing sufficient to promote a cooperative effect of thermal energy between the scan lines.

A system capable of performing the methods herein described is shown in FIG. 6. The system allows for the coupling of a high intensity focused ultrasound transducer to a patient. The system has a fluid circuit 20 for conveying a coupling fluid F between the coupling reservoir 28 contained within a transducer housing 29 and a vacuum chamber 24. The fluid F is moved through the circuit using a pump 22. A vacuum chamber 24 serves to degas the fluid F. A chiller 30 may optionally be connected to the fluid circuit 20 to keep the fluid F cold. A filter 26 is also provided for removing particulate matter from the fluid. The coupling reservoir 28 provides a fluid environment in which the transducer is suspended. The fluid serves as an internal coupling agent allowing the ultrasound energy emitted from the transducer to reach the patient skin surface with as little attenuation and signal loss as possible. The system described provides degassing and filtering so the fluid is free from matter that that might cause particulate nuclei induced cavitation (caviation of the fluid caused by interaction between the dissolved gasses or particles suspended in the fluid, and the ultrasound energy emitted from the transducer). More detailed descriptions of the therapy head having a coupling reservoir are described in co-pending application Ser. Nos. 11/027,912; entitled “Ultrasound Therapy Head with Movement Control,” and 11/026,519; entitled “Systems and Methods for the Destruction of Adipose Tissue” and U.S. patent application Ser. No. 11/027,491; entitled “Disposable Transducer Seal.” All three applications being filed on Dec. 29, 2004.

Various parameters in the system can be used to achieve differing E_{f} values, and thus different clinical results. Although two procedures may have the same E_{f} value, they can have substantially different results in tissue. For instance, at a lower E_{f} value one therapy can generate substantial mechanical and thermal effects in tissue, causing cellular disruption and a substantial wound healing response. The same E_{f} value therapy may be modified in the variable so that a relatively modest thermal reaction is achieved which produces a milder clinical effect and causes a less dramatic wound healing response. Thus one provides for the destruction of adipose tissue, while the other initiates a natural process by which adipose tissue volumes are reduced.

While various embodiments have been shown and described herein, it should be apparent to those skilled in the art that such embodiments are provided by way of example only. Numerous variations, changes, and substitutions will now occur to those skilled in the art without departing from the spirit of the invention. It should be understood that various alternatives to the embodiments as described herein may be employed in practicing the invention. It is intended that the following claims define the scope of the invention and that methods and structures within the scope of these claims and their equivalents be covered thereby.

What is claimed is:

1. A method for projecting a volume of tissue onto a skin surface in preparation for a noninvasive cosmetic therapy procedure, the method comprising the steps of:
determining a volume of tissue suitable for a noninvasive cosmetic therapy procedure; and
creating a surface area map corresponding to said volume of tissue on a skin surface wherein said surface area map provides sufficient volumetric information to guide a user in conducting said noninvasive cosmetic therapy procedure.

2. The method of claim 1, wherein said creating step further comprises dividing said surface area into a plurality of individual treatment sections.

3. The method of claim 1, wherein said non-invasive cosmetic therapy procedure uses high intensity ultrasound.

4. The method of claim 1, wherein said non-invasive cosmetic therapy procedure uses high intensity focused ultrasound.

5. The method of claim 1, wherein the determining step further comprises scanning a volume of tissue using an imaging device.

6. A method for initiating a reduction in a volume of adipose tissue comprising the step of:
moving a therapeutic high intensity ultrasound transducer over a patient skin surface while emitting high intensity ultrasound into a volume of adipose tissue such that a biological response is initiated that leads to a reduction in said volume of adipose tissue.

7. The method of claim 6, wherein the applying step is done by continuous sweeps of the energy applicator.

8. The method of claim 6, wherein the applying step is done by position jumping of said energy applicator.

9. The method of claim 6, wherein the applying step is done by time delay jumping of said energy applicator.

10. The method of claim 6, wherein the transducer is a high intensity focused ultrasound transducer.

11. A method for reducing a volume of adipose tissue in a patient comprising the steps of:
moving a high intensity ultrasound transducer over a skin surface; and
irradiating a volume of adipose tissue below said skin surface using said high intensity ultrasound transducer wherein said transducer deposits an energy flux value of at least 35 J/cm².

12. The method of claim 11, wherein the transducer is capable of depositing an energy flux value up to 456 J/cm².

13. The method of claim 11, wherein the transducer is moved over a patient surface in a continuous sweeping motion.

14. The method of claim 11, wherein the transducer is moved over the patient body in a jumping manner.

15. A method of destroying adipose tissue using high intensity focused ultrasound, the method comprising the steps of:
determining a volume of adipose tissue to be treated;
marking out a corresponding surface area of skin;
dividing the surface area into a plurality of individual treatment sections;
applying therapeutic ultrasound energy to one section of said plurality of individual treatment sections with an ultrasound transducer until sufficient energy has been deposited to at least partially destroy the adipose tissue.

16. The method of claim 15, wherein the step of applying therapeutic ultrasound energy further comprises moving said ultrasound transducer in a manner such that sequential applications of ultrasound energy are spaced apart to non-adjacent sections.

17. The method of claim 15, wherein the step of applying therapeutic ultrasound energy further comprises providing a timing delay to the treatment of a physically adjacent section.

18. The method of claim 15, wherein the step of applying therapeutic ultrasound energy further comprises scanning a therapy transducer across said surface area at a velocity and line spacing sufficient to promote a cooperative effect of thermal energy build up in said adipose tissue.

19. The method of claim 15, wherein the step of applying therapeutic ultrasound energy further comprises creating an energy flux level in the adipose tissue in excess of 35 J/cm².

20. A system for coupling a high intensity focused ultrasound transducer to a patient comprising:
a fluid circuit for conveying a coupling fluid;
a pump for circulating coupling fluid through the fluid circuit;
a vacuum chamber connected to apply a pressure gradient to said fluid circuit such that dissolved gasses are drawn out of said coupling fluid;
a filter for removing particulate matter; and
a coupling reservoir connected to said circuit for coupling a transducer to a patient.
